(19) United States
(12) Patent Application Publication
(10) Pub. No.: US 2003/0006379 A1
(43) Pub. Date: Jan. 9, 2003

(54) Target container for neutron scattering apparatus

(76) Inventors: Ryutaro Hino, Naka-gun (JP); Masanori Kaminaga, Naka-gun (JP); Syuichi Ishikura, Kobe (JP); Masayuki Uzawa, Kobe (JP); Toru Iitsuka, Kobe (JP)

Correspondence Address: WENDEROTH, LIND & PONACK, L.L.P., 2033 K STREET N. W., SUITE 800, WASHINGTON, DC, 20006-1021, US

(21) Appl. No.: 10/019,960

(22) PCT Filed: May 25, 2001
(86) PCT No.: PCT/JP01/04407

(30) Foreign Application Priority Data
May 26, 2000 (JP) 2000-156197
Apr. 4, 2001 (JP) 2001-105801

(51) Int. Cl. G21G 5/00; A61N 5/00

(52) U.S. Cl. 250/492.1

(57) ABSTRACT

Target cell of a neutron scattering device, containing liquid metal target material, has its beam window, through which proton beam enters, formed in a structure that is thin and yet can stand both pressure wave stress and thermal stress. In the target cell comprising an outer casing and an inner casing, both having a front face formed with the beam window, and being formed in a double structure of the outer casing and the inner casing with a predetermined distance maintained therebetween so that cooling medium is supplied there and target material is supplied into the inner casing, the beam window of the inner casing is made in a flat plate structure. By this flat plate structure, its rigidity is lowered and cell stress of a secondary stress character caused in the target cell by the pressure wave is reduced. Or, the beam window of the inner casing is formed to have a linear front face in the vertical section and a continuously curved front face in the horizontal section. Thereby, in the beam window of the inner casing, the pressure wave stress and the thermal stress are coped with by the linear front face and the continuously curved front face, respectively.

### TECHNICAL FIELD

The present invention relates generally to a target cell of a neutron scattering device and more particularly to a target cell containing target material, such as liquid metal, in a neutron scattering device using proton beam of as large a strength as about 1 MW or more.

### BACKGROUND ART

A neutron scattering device is a device for generating neutron beam by a nucleus breaking action caused by proton beam discharged from an accelerator to enter heavy metal. This device is used for performing advanced studies in the wide range of life science, materials science, nuclear physics, etc.

In the neutron scattering device using proton beam especially of a large strength, mainly from the viewpoint of an irradiation deterioration due to protons and neutrons, not solid metal but liquid metal, such as mercury, lead or lead-bismuth alloy, as target material, is employed and therein the liquid metal itself as the target material is circulated via a cooling system so that heat generated by the nucleus breaking action may be removed.

While a target cell used in such a neutron scattering device is currently being developed by trials and errors, its basic structure and outline of main portions will be described below with reference to FIGS. 3(a) and (b) and FIGS. 4(a) and (b).

FIGS. 3(a) and (b) are schematic views showing a basic structure of a target cell in the prior art, wherein FIG. 3(a) is a longitudinal horizontal cross sectional view and FIG. 3(b) is a longitudinal vertical cross sectional view. FIGS. 4(a) and (b) are schematic enlarged views of a front end portion of another target cell in the prior art, wherein FIG. 4(a) is a longitudinal horizontal cross sectional view and FIG. 4(b) is a longitudinal vertical cross sectional view.

In FIGS. 3(a) and (b) numeral 1 designates an outer casing, that has its front face formed in a semi-cylindrical shape, as understood from the figures, and this front face has a portion forming a beam window 1a through which proton beam enters the device of the target cell.

Numeral 2 designates an inner casing, that is arranged within the outer casing 1 so as to form a cooling space 3 having a predetermined width maintained between the outer casing 1 and the inner casing 2, thereby forming a double structure in which the inner casing 2 is coaxially arranged in the outer casing 1. A front face of the inner casing 2 is likewise formed in a semi-cylindrical shape so as to be arranged in parallel with the beam window 1a of the outer casing 1 and also has a portion forming a beam window 2a.

Through the cooling space 3 formed between the outer casing 1 and the inner casing 2, when cooling is needed, cooling medium of distilled water or the like is supplied from upstream and, when no cooling is needed, inert gas of helium or the like is supplied so that the device may be maintained in a stable state.

In an internal space 4 of the inner casing 2, there are arranged a plurality of partition plates 5 extending in the longitudinal direction of the inner casing 2. Target material 8, as shown by arrows in FIG. 3(a), that is selected from liquid metals of mercury, lead, lead-bismuth alloy, etc. is supplied into the inner casing 2 from a supply pipe 6 on the upstream side to flow therein in a slow velocity of about 1 m/sec. and then to turn, as shown by the arrows, at the position of the beam window 2a and is thereafter returned into a recovery pipe 7 on the downstream side.

Thus, when proton beam is discharged from an accelerator (not shown), arranged in the front of the target cell, to enter the target material 8 via the beam window 1a of the outer casing 1 and the beam window 2a of the inner casing 2, a nucleus breaking action occurs in the target material 8 to generate neutrons.

In the basic structure of the target cell constructed as above, as a main portion thereof, there is considered a concrete structure of beam windows 1a, 2a of an outer casing 1 and an inner casing 2, as shown in a prior art example of FIGS. 4(a) and (b).

In the target cell of FIGS. 4(a) and (b), a front face of the outer casing 1 is formed in an approximately hemispherical shape, as understood from the figures, and other portions are basically the same as those shown in FIGS. 3(a) and (b).

The beam window 2a of the inner casing 2, as seen in FIG. 4(a), has its both side corner faces formed in a curved face having a curvature R₂ and its front face formed in a linear face. The beam window 2a, as seen in FIG. 4(b), including the portions of the curved face and the linear face, has its front face formed in a curved face having a curvature R₃.

Thus, when proton beam H discharged from the front side passes through the beam window 1a of the outer casing 1 and the beam window 2a of the inner casing 2 to enter target material 8 that is circulating in an internal space of the inner casing 2, a nucleus breaking action occurs in the target material 8 to generate neutrons.

Also, through a cooling space 3 formed between the outer casing 1 and the inner casing 2, cooling medium 9 of distilled water or the like is supplied, so that balance adjustment between cooling of the heat of reaction in the inner casing 2 and pressure in the inner casing 2 is effected.

### DISCLOSURE OF THE INVENTION

In the neutron scattering device as mentioned above, it is considered so that, when the proton beam H enters, the liquid metal makes thermal expansion to cause pressure wave of about 100 MPa and so the device is basically needed to be of a structure to stand such a large force.

On the other hand, the structure of the beam windows through which the proton beam H enters the liquid metal in the target cell is needed to be made thinner in the cooling point of view and hence it is preferable to make the design of the beam windows that are appropriate to satisfy the cooling condition, while a soundness against the pressure wave is maintained.

In view of the mentioned circumstances, it is an object of the present invention to provide a target cell of a neutron scattering device in which beam windows of the target cell through which proton beam enters is made in such a thinned structure as needed in the cooling point of view as well as in such a structure as stands both of pressure wave stress and thermal stress.

In order to achieve the mentioned object, the present invention provides the following first to fourth means:

As the first means, in a target cell of a neutron scattering device comprising an outer casing and an inner casing, each of which has a front face formed with a beam window through which proton beam enters, and being formed in a double structure in which the inner casing is arranged within the outer casing with a predetermined distance being maintained therebetween so that cooling medium may be supplied between the outer casing and the inner casing and target material may be supplied into the inner casing, the beam window of the inner casing is made in a flat plate structure.

According to the present first means, the inner casing arranged within the outer casing to form the double structure has its front face formed with the beam window, that is made in the flat plate structure, and hence rigidity of this beam window can be lowered, cell stress having a character of secondary stress is reduced and a thin structure, that makes cooling thereof easier, can be obtained.

As the second means, the target cell of the first means is made in a differential pressure structure in which pressure in the outer casing is made higher than in the inner casing.

According to the present second means, the beam window of the front face of the inner casing arranged within the outer casing is formed in the flat plate structure and, in addition thereto, the pressure in the outer casing, that is separated from the inner casing by the beam window of the inner casing, is made higher than the pressure in the inner casing. Hence, the pressure wave caused in the inner casing is coped with also by the cooling medium supplied into the outer casing and the beam window of the flat plate structure can stand also bending stress to be maintained in a sound state.

As the third means, in a target cell of a neutron scattering device comprising an outer casing and an inner casing, each of which has a front face formed with a beam window through which proton beam enters, and being formed in a double structure in which the inner casing is arranged within the outer casing with a predetermined distance being maintained therebetween so that cooling medium may be supplied between the outer casing and the inner casing and target material may be supplied into the inner casing, the beam window of the inner casing has a front face formed in a linear face as seen in a longitudinal vertical cross section of the inner casing and in a continuously curved face as seen in a longitudinal horizontal cross section of the inner casing.

According to the present third means, the beam window of the inner casing has its front face formed in the linear face as seen in the longitudinal vertical cross section of the inner casing and in the continuously curved face as seen in the longitudinal horizontal cross section of the inner casing. Hence, as to the pressure wave stress and the thermal stress caused to act on the beam window of the inner casing by the proton beam entering the target material, the former pressure wave stress is coped with by the linear face in the longitudinal vertical cross section and the latter thermal stress is coped with by the continuously curved face in the longitudinal horizontal cross section, so that both of the stresses are mitigated to be suppressed within an appropriate range.

As the fourth means, in the target cell of the third means, a curvature of the continuously curved face is about 2.5 to 0.8 times of a width of the inner casing as seen in the longitudinal horizontal cross section of the inner casing.

According to the present fourth means, with respect to the beam window of the inner casing constructed by the continuously curved face in the longitudinal horizontal cross section of the inner casing, where the width of the inner casing is taken as a reference, the curvature of the continuously curved face is constructed to be about 2.5 to 0.8 times of the width of the inner casing. Thereby, both of the pressure wave stress and the thermal stress acting on the beam window of the inner casing can be suppressed within an appropriate range.

### BRIEF DESCRIPTION OF THE DRAWINGS

FIGS. 1(a) and (b) are schematic enlarged views of a front end portion as a main portion of a target cell in a neutron scattering device of a first embodiment according to the present invention, wherein FIG. 1(a) is a longitudinal horizontal cross sectional view and FIG. 1(b) is a longitudinal vertical cross sectional view.

FIGS. 2(a) and (b) are schematic enlarged views of a front end portion as a main portion of a target cell in a neutron scattering device of a second embodiment according to the present invention, wherein FIG. 2(a) is a longitudinal horizontal cross sectional view and FIG. 2(b) is a longitudinal vertical cross sectional view.

FIGS. 3(a) and (b) are schematic views showing a basic structure of a target cell in the prior art, wherein FIG. 3(a) is a longitudinal horizontal cross sectional view and FIG. 3(b) is a longitudinal vertical cross sectional view.

FIGS. 4(a) and (b) are schematic enlarged views of a front end portion of another target cell in the prior art, wherein FIG. 4(a) is a longitudinal horizontal cross sectional view and FIG. 4(b) is a longitudinal vertical cross sectional view.

### BEST MODE FOR CARRYING OUT THE INVENTION

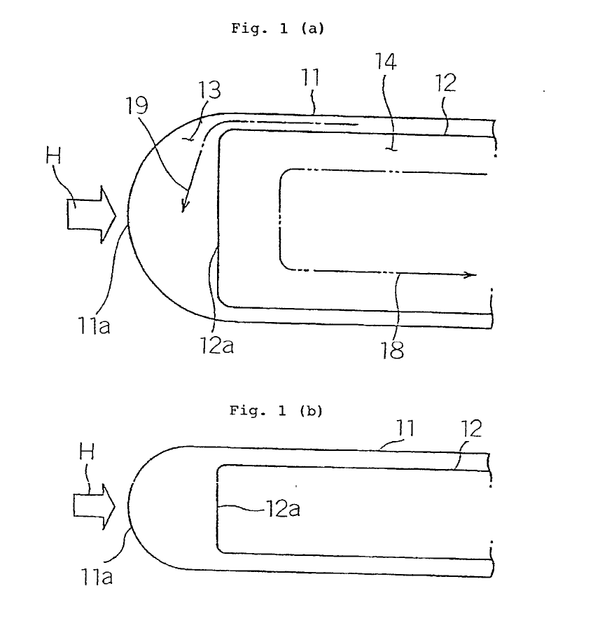

A first embodiment according to the present invention will be described with reference to FIGS. 1(a) and (b).

FIGS. 1(a) and (b) are schematic enlarged views of a front end portion as a main portion of a target cell in a neutron scattering device of the first embodiment, wherein FIG. 1(a) is a longitudinal horizontal cross sectional view and FIG. 1(b) is a longitudinal vertical cross sectional view.

In the present first embodiment, numeral 11 designates an outer casing, that forms an outer shell of the target cell in the neutron scattering device A front face of the outer casing 11 is formed in an approximately hemispherical shape and has a portion forming a beam window 11a through which proton beam H, discharged from an accelerator (not shown), enters.

Numeral 12 designates an inner casing, that is arranged within the outer casing 11 so as to form a cooling space 13 having a predetermined width maintained between the outer casing 11 and the inner casing 12, thereby forming a double structure in which the inner casing 12 is coaxially arranged in the outer casing 11. A front face of the inner casing 12 is arranged opposingly to the beam window 11a within the outer casing 11 and has a portion forming a beam window 12a.

Unlike the beam window 11a of the outer casing 11 that is formed in the approximately hemispherical shape. the beam window 12a of the inner casing 12 is formed in a flat plate structure in which the proton beam H, having passed through the beam window 11a of the outer casing 11, passes through the flat plate orthogonally.

Through the cooling space 13 formed between the outer casing 11 and the inner casing 12, cooling medium 19 of distilled water or the like is supplied from upstream. Also, in an internal space 14 of the inner casing 12, target material 18, selected from liquid metals of mercury, lead, lead-bismuth alloy, etc. is supplied to flow therein in a slow velocity of about 1 m/sec. Where pressure in the cooling space 13 is Po, that in the inner casing 14 is Pi and differential pressure thereof is ΔP=Po−Pi, a relation of ΔP>0 is maintained.

In the present first embodiment constructed as mentioned above, when proton beam is discharged from an accelerator (not shown), arranged in the front of the target cell, to enter the target material 18 via the beam window 11a of the outer casing 11 and the beam window 12a of the inner casing 12, a nucleus breaking action occurs in the target material 18, thereby scattering neutrons and, at the same time, causing the internal pressure wave.

Generally, in a vessel or cell, like the target cell of the present embodiment, that is pressurized internally by the internal pressure wave or the like, as a pressure design, a spherical structure is the strongest one, then a cylindrical structure is preferable and a flat plate structure will be the weakest.

However, the inventors here, having put eyes on the fact that the cell stress caused in the target cell by the pressure wave has a character like a secondary stress, have obtained an expertise that, if, as a member that is usually put in the severest stress condition, the beam window 12a of the inner casing 12 through which the proton beam H enters is made not in a hemispherical or semi-cylindrical structure but rather in a flat plate structure, then the rigidity of, and around, that member would be lowered to thereby also reduce the stress caused in the cell, and it was concluded that such construction can be advantageously employed.

Also, according to this flat plate structure, membrane stress that is peculiar to the spherical or cylindrical structure can be reduced to ½ or less, thereby making it possible to facilitate the design and to reduce the plate thickness to ½ and thus the thermal stress also can be largely reduced.

It is to be noted that, by making the beam window 12a of the inner casing 12 in the flat plate structure, the bending stress becomes larger than that in the case of the hemispherical or semi-cylindrical structure but, as a countermeasure therefor, an outer pressure design is employed in which the outer pressure is made higher by several bars, 3 to 4 bars or 4 to 5 bars for example, than the inner pressure so as to ensure the stress to set off the pressure caused by the pressure wave and thereby the bending stress can be reduced to an allowable stress level.

Also, if the point of the outer pressure design only is considered, even the hemispherical or semi-cylindrical structure could theoretically satisfy the stress reduction by the mentioned outer pressure design. But usually, as the mentioned hemispherical or semi-cylindrical structure is of a high strength for the inner pressure, in order to reduce the stress to a substantial extent, it is necessary to form a differential pressure of several MPa, which is not practical.

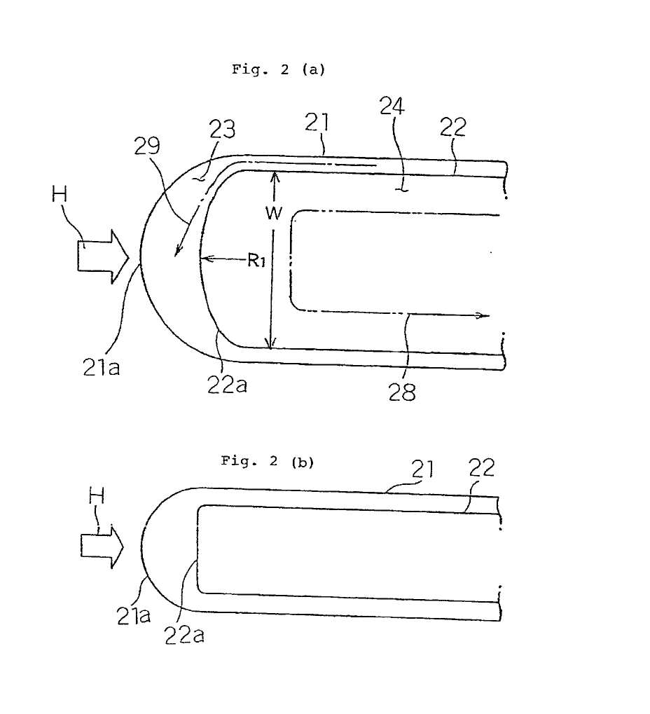

Next, a second embodiment according to the present invention will be described with reference to FIGS. 2(a) and (b).

FIGS. 2(a) and (b) are schematic enlarged views of a front end portion as a main portion of a target cell in a neutron scattering device of the second embodiment, wherein FIG. 2(a) is a longitudinal horizontal cross sectional view and FIG. 2(b) is a longitudinal vertical cross sectional view.

In the present second embodiment, numeral 21 designates an outer casing, that forms an outer shell of the target cell in the neutron scattering device. A front face of the outer casing 21 is formed in an approximately hemispherical shape and has a portion forming a beam window 21a through which proton beam H, discharged from an accelerator (not shown), enters.

Numeral 22 designates an inner casing, that is arranged within the outer casing 21 so as to form a cooling space 23 having a predetermined width maintained between the outer casing 21 and the inner casing 22, thereby forming a double structure in which the inner casing 22 is coaxially arranged in the outer casing 21. A front face of the inner casing 22 is arranged opposingly to the beam window 21a within the outer casing 21 and has a portion forming a beam window 22a.

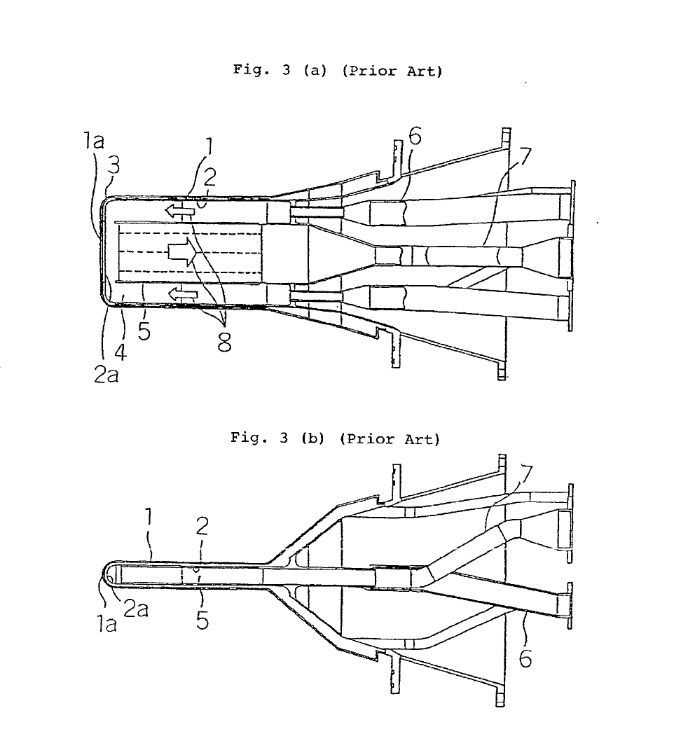

Unlike the beam window 2a of the inner casing 2 that has its front face formed in the semi-cylindrical shape, as described with respect to FIGS. 3(a) and (b), and also unlike the beam window 12a of the first embodiment that is formed in the flat plate structure, as described with respect to FIGS. 1(a) and (b), the beam window 22a of the inner casing 22 has its front face formed in a linear face, as seen in FIG. 2(b), and in a continuously curved face, as seen in FIG. 2(a).

Further, through the cooling space 23 formed between the outer casing 21 and the inner casing 22, cooling medium 29 of distilled water or the like is supplied from upstream. Also, in an internal space 24 of the inner casing 22, target material 28, selected from liquid metals of the mercury, lead, lead-bismuth alloy, etc. is supplied to flow therein in a slow velocity of about 1 m/sec.

In the present second embodiment constructed as mentioned above, when proton beam is discharged from an accelerator (not shown), arranged in the front of the target cell, to enter the target material 28 via the beam window 21a of the outer casing 21 and the beam window 22a of the inner casing 22, a nucleus breaking action occurs in the target material 28, thereby scattering neutrons and, at the same time, causing the internal pressure wave. Thus, the pressure wave stress caused by the pressure wave and the thermal stress following the nucleus breaking action act on the beam window 22a.

Here, experiments were carried out by the inventors here for measuring the thermal stress and the pressure wave stress acting on the beam window 22a, wherein a width W in the longitudinal horizontal cross section of the inner casing 22 of the present second embodiment was set to 600 mm and, thereto, a curvature R₁ of the curved front face in the longitudinal horizontal cross section of the beam window 22a was variously changed.

As for the thermal stress, it was confirmed that, for the curvature R₁ set to 1600 mm, the membrane stress was 160 MPa and the bending stress was 211 MPa. For the curvature R₁ set to 800 mm, the membrane stress was 123 MPa and the bending stress was 197 MPa. Also, for the curvature R₁ set to 500 mm, the membrane stress was 90 MPa and the bending stress was 182 MPa.

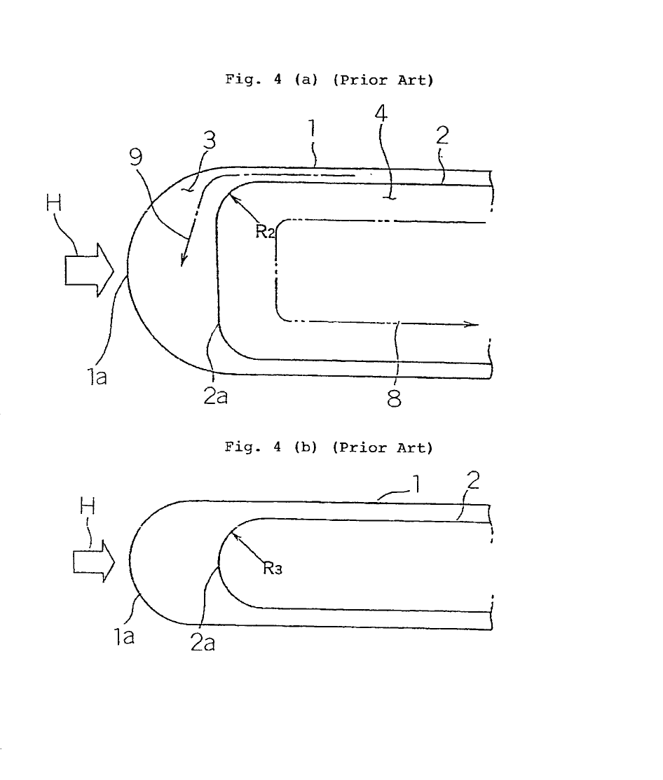

For the purpose of comparison, like measurements were carried out for the target cell as described with respect to FIGS. 4(a) and (b), which resulted in the membrane stress of 143 MPa and the bending stress of 193 MPa. Also, the measurements were done for the target cell of the mentioned first embodiment as described with respect to FIGS. 1(a) and (b), which resulted in the membrane stress of 188 MPa and the bending stress of 220 MPa.

Also, as for the pressure wave stress in the present second embodiment, it was confirmed that, for the curvature R₁ set to 1600 mm, the membrane stress was 70 MPa and the bending stress was 326 MPa. For the curvature R₁ set to 800 mm, the membrane stress was 70 MPa and the bending stress was 326 MPa, both of which were the same as in the case of the curvature R₁ of 1600 mm. Also, for the curvature R₁ set to 500 mm, the membrane stress was 72 MPa and the bending stress was 295 MPa.

Like the case of the thermal stress, for the purpose of comparison, measurements were done for the target cell as described with respect to FIGS. 4(a) and (b), which resulted in the membrane stress of 146 MPa and the bending stress of 483 MPa. Also, with respect to the target cell of the first embodiment of FIGS. 1(a) and (b), the membrane stress was 71 MPa and the bending stress was 333 MPa.

From the above, the following finding was obtained, that is, for the width W of 600 mm in the longitudinal horizontal cross section of the inner casing 22, if the curvature R₁ is set to 1600 mm, that is about 2.5 times of the width W of 600 mm, the membrane stress due to the thermal stress comes close to its limit. Thus, around this area of the ratio of the curvature R₁ to the width W of about 2.5, there is an upper limit of the curvature. Also, if the curvature R₁ is set to 500 mm, that is about 0.8 times of the width W of 600 mm, both of the thermal stress and the pressure wave stress come in a satisfactory range ensuring a safety. While a still smaller curvature is preferable to be pursued, if a neutronics effect is taken account of, it will be reasonably considered that a lower limit of the curvature exists around the area of the ratio of the curvature R₁ to the width W of about 0.8

If additionally explained, in this kind of the neutron scattering device, distribution of the neutron generation depends on a passing distance of the proton beam H in the target material such as mercury. In order to make a sharp distribution peak, as the proton beam H to enter is a uniform beam, it is necessary to arrange the passing distance uniformly in the target material. On the other hand, as the curvature R₁ is made smaller, the passing distance of the proton beam H in the target material becomes different between the central portion and the peripheral portion of the target cell and the distribution peak of the neutrons becomes wider. For this reason, that is, for the neutronics effect, the lower limit of the curvature is regulated.

Thus, according to the present second embodiment, by forming the front face of the beam window 22a of the inner casing 22 in the linear face as seen in the longitudinal vertical cross section of the inner casing, the same strength against the pressure wave stress is ensured as in the flat plate structure described with respect to the first embodiment of FIGS. 1(a) and (b). In addition thereto, by forming the front face of the beam window 22a of the inner casing 22 in the continuously curved face as seen in the longitudinal horizontal cross section, especially by making the curvature R₁ of the curved face about 2.5 to 0.8 times of the width W of the inner casing 22, such a structure as to meet the thermal stress by letting the thermal stress escape along this curved face is realized and hence an inner casing having a higher safety, such as the inner casing 22, can be obtained.

While the preferred forms of the present invention have been described, it is to be understood that the invention is not limited to the particular construction and arrangement herein illustrated and described but embraces such modified forms thereof as come within the scope of the appended claims.

### INDUSTRIAL APPLICABILITY

According to the invention as set forth in claim 1, in a target cell of a neutron scattering device comprising an outer casing and an inner casing, each of which has a front face formed with a beam window through which proton beam enters, and being formed in a double structure in which the inner casing is arranged within the outer casing with a predetermined distance being maintained therebetween so that cooling medium may be supplied between the outer casing and the inner casing and target material may be supplied into the inner casing, the beam window of the inner casing is made in a flat plate structure. Thus, in the inner casing arranged within the outer casing to form the double structure, the beam window of the inner casing is made in the flat plate structure, and hence rigidity of this beam window can be lowered, cell stress having a character of secondary stress is reduced and a thin structure, that makes cooling thereof easier, can be obtained. Thereby, a target cell of a neutron scattering device that has a high and appropriate practicality can be obtained.

According to the invention as set forth in claim 2, the target cell of claim 1 is made in a differential pressure structure in which pressure in the outer casing is made higher than in the inner casing. Thus, in addition to the fact that the beam window of the front face of the inner casing arranged within the outer casing is formed in the flat plate structure, the pressure in the outer casing, that is separated from the inner casing by the beam window of the inner casing, is made higher than the pressure in the inner casing. Hence, the pressure wave caused in the inner casing is coped with also by the cooling medium supplied into the outer casing and the beam window of the flat plate structure can stand also bending stress to be maintained in a sound state. Thereby, a target cell of a neutron scattering device that has a high and appropriate practicality can be obtained.

According to the invention as set forth in claim 3, in a target cell of a neutron scattering device comprising an outer casing and an inner casing, each of which has a front face formed with a beam window through which proton beam enters, and being formed in a double structure in which the inner casing is arranged within the outer casing with a predetermined distance being maintained therebetween so that cooling medium may be supplied between the outer casing and the inner casing and target material may be supplied into the inner casing, the beam window of the inner casing has a front face formed in a linear face as seen in a longitudinal vertical cross section of the inner casing and in a continuously curved face as seen in a longitudinal horizontal cross section of the inner casing. Thus, in the beam window of the inner casing having its front face formed in the linear face as seen in the longitudinal vertical cross section of the inner casing and in the continuously curved face as seen in the longitudinal horizontal cross section of the inner casing, the stress caused therein is coped with as follows. That is, as to the pressure wave stress and the thermal stress caused to act on the beam window of the inner casing by the proton beam entering the target material, the pressure wave stress is coped with by the linear face in the longitudinal vertical cross section and the thermal stress is coped with by the continuously curved face in the longitudinal horizontal cross section, so that both of the stresses are mitigated to be suppressed within an appropriate range. Thereby, a target cell of a neutron scattering device that has a high and appropriate practicality can be obtained.

According to the invention as set forth in claim 4, in the target cell of claim 3, a curvature of the continuously curved face is about 2.5 to 0.8 times of a width of the inner casing as seen in the longitudinal horizontal cross section of the inner casing. Thus, with respect to the beam window of the inner casing constructed by the continuously curved face in the longitudinal horizontal cross section of the inner casing, where the width of the inner casing is taken as a reference, the curvature of the continuously curved face is constructed to be about 2.5 to 0.8 times of the width of the inner casing. Hence, both of the pressure wave stress and the thermal stress acting on the beam window of the inner casing can be suppressed within an appropriate range. Thereby, a target cell of a neutron scattering device that has a high and appropriate practicality can be obtained.

What is claimed is:

1. A target cell of a neutron scattering device comprising an outer casing and an inner casing, each of which has a front face formed with a beam window through which proton beam enters, and being formed in a double structure in which said inner casing is arranged within said outer casing with a predetermined distance being maintained therebetween so that cooling medium may be supplied between said outer casing and said inner casing and target material may be supplied into said inner casing, wherein said beam window of said inner casing is made in a flat plate structure.

2. A target cell of a neutron scattering device as claimed in claim 1, wherein said target cell is made in a differential pressure structure in which pressure in said outer casing is made higher than in said inner casing.

3. A target cell of a neutron scattering device comprising an outer casing and an inner casing, each of which has a front face formed with a beam window through which proton beam enters, and being formed in a double structure in which said inner casing is arranged within said outer casing with a predetermined distance being maintained therebetween so that cooling medium may be supplied between said outer casing and said inner casing and target material may be supplied into said inner casing, wherein said beam window of said inner casing has a front face formed in a linear face as seen in a longitudinal vertical cross section of said inner casing and in a continuously curved face as seen in a longitudinal horizontal cross section of said inner casing.

4. A target cell of a neutron scattering device as claimed in claim 3, wherein a curvature of said continuously curved face is about 2.5 to 0.8 times of a width of said inner casing as seen in the longitudinal horizontal cross section of said inner casing.
